(19) United States
(12) Patent Application Publication
(10) Pub. No.: US 2021/0176862 A1
(43) Pub. Date: Jun. 10, 2021

(54) Feedthrough Comprising Interconnect Pads

(71) Applicant: Morgan Advanced Ceramics, Inc., New Bedford, MA (US)

(72) Inventors: Abhishek S. Patnaik, Lexington, MA (US); John Antalek, East Freetown, MA (US)

(73) Assignee: Morgan Advanced Ceramics, Inc., New Bedford, MA (US)

(21) Appl. No.: 17/116,150

(22) Filed: Dec. 9, 2020

Related U.S. Application Data
(60) Provisional application No. 62/946,115, filed on Dec. 10, 2019.

(30) Foreign Application Priority Data
Jan. 28, 2020 (GB) 2001185.4
Jul. 16, 2020 (GB) 2010951.8

(51) Int. Cl. H05K 1/11; H05K 3/38; H05K 1/09

(52) U.S. Cl. CPC H05K 1/113; A61N 1/3754; H05K 1/09; H05K 3/388

(57) ABSTRACT

A feedthrough assembly (1) comprising a feedthrough body (10) comprising: a ceramic body (2) having a first side (3) and a second side (4); a conductive element (5) extending through said ceramic body (2) between said first side (3) and said second side (4); a conductive pad (6) electrically connected to said conductive element (5). The conductive pad (6) comprises a multi-layered arrangement comprising: a bonding layer (7) comprising one or more elements selected from the group consisting of Ti, Zr, Nb and V, said bonding layer in bonding contact with an end of the conductive element and the first or second side of the ceramic body; and at least one of a diffusion barrier layer (8) directly disposed upon said bonding layer, comprising one or more elements selected from the group consisting of Nb, Ta, W, Mo and nitrides thereof, and at least one of (i) said diffusion layer having a different composition than the bonding layer; and (ii) one or more sealing layers (9, 9a, 9b), disposed upon said diffusion barrier layer.

### CROSS-REFERENCE TO RELATED APPLICATIONS

This application claims priority to U.S. Provisional Application No. 62/946,115, filed Dec. 10, 2019, United Kingdom Application No. 2001185.4, filed Jan. 28, 2020, and United Kingdom Application No. 2010951.8, filed Jul. 16, 2020, the entire disclosures of which are hereby incorporated by reference herein.

### FIELD

The present disclosure relates to feedthroughs comprising interconnect pads and methods of producing thereof. In particular, the present disclosure relates to implantable medical devices comprising said feedthroughs spaced in close proximity.

### BACKGROUND

Assemblies comprising metal and ceramic components are used in a wide range of applications. Ceramic-metal assemblies have found particular use in feedthroughs, where one or more electrical conductors are required to pass through a ceramic insulator to provide one or more electrically conductive connections from one surface of the ceramic insulator to another surface of the ceramic insulator. Such arrangements are widely used, for example, in aerospace, transportation, communication and power tube (e.g. x-ray, radio frequency) and medical applications; the present disclosure is not limited to any one application.

Electronic biomedical implants are being used increasingly to diagnose, prevent and treat diseases and other medical conditions. Implantable electronic devices must necessarily comply with safety standards before being approved for clinical use; for example, such implanted devices are needed to be housed in hermetic packages that incorporate electrical feedthroughs for signal transfer between the housed electronic device and the environment. By encapsulating electronically active components hermetically, the human or animal body is protected from toxicity of conventional electronic components and the device is also protected from the relatively harsh environment of the body that may otherwise cause the device to fail prematurely. Such implantable devices, especially those that interface with the human nervous system or organs in the human body such as the cochlea or the retina require a multiplex of electrical leads in the small confined space of a miniature feedthrough. Ceramic materials such as alumina or metals such as titanium have a long history of success in bionic feedthroughs in devices including pace-makers and cochlear implants. Biocompatible ceramic-metal feedthrough systems may be considered to be the most reliable choice of materials for such devices owing to their chemical inertness (e.g. biocompatibility) and longevity (e.g. bio-stability).

The application of electronic biomedical implants in interacting with the human nervous system is becoming increasingly complex, particularly in neural prosthesis where high resolution stimulating or recording arrays are positioned near peripheral nerves or in the brain. Densely packed electrical feedthroughs are needed to carry input/output (I/O) signals to and from these implanted devices. For certain therapies, it is desirable to increase the number of electrical conductors (which have many names in the art of feedthroughs including: leads, pathways, pins, wires, and vias) in the feedthroughs to increase the overall number of I/O signals to meet the demands of these critical applications.

The challenge to provide densely packed electrical feedthroughs is met with the dimensional constraints placed on reducing the overall size of the feedthrough since it is undesirable to implant large devices (including a large feedthrough) in the human or animal body. In particular, it is also desirable to reduce the invasiveness of the implantation surgeries and/or the nature of the placement of the device for the target therapies such as in retinal implants where the nature of the application necessitates only those devices that are suitably small. When the device design requires both a large number of conductors (i.e. high pin count) and a small-sized feedthrough, conventional feedthrough manufacturing techniques are inadequate and no longer viable. Existing technologies have limits as to the spacing of conductors within the feedthrough which inhibits the ability to increase the density of conductors in the feedthroughs. Therefore, until now, it has been necessary to opt either to make larger feedthroughs thereby increasing the size of the overall device comprising the feedthrough in order to accommodate a higher density of conductors or to reduce the density of conductors thereby limiting number of I/O signals in favour of a smaller-sized feedthrough, both of which fail to meet industrial demands.

The compressive force imparted onto conductors embedded in the ceramic matrix of a feedthrough during co-sintering is often relied upon for hermeticity. The interfaces between the conductors and the ceramic body may lack the hermeticity requirements demanded for suitable feedthroughs in critical and high performance applications.

As active implants are miniaturized to reduce trauma of invasive surgeries, the pin to pin distance between the conductors also decreases. When the pin to pin distance is very small, it becomes impossible to assemble the feedthroughs by gold brazing. Therefore, many investigators have proposed alternative methods to manufacture metal feedthroughs In such cases the ceramic shrinks directly into the conductor, the bonding may not be as strong as traditional gold brazing which may lead to lack of hermeticity due to slippage at the surface. Prior art methods use ceramic and metal powders to create the conductor path in the feedthrough by screen printing or filling holes in green ceramic, then co-firing the whole body to get sintered feedthrough. In such methods the metal ceramic powders in the ceramic-metal composite (CMC) paste intimately bond with walls of the ceramic vias and give good hermeticity. The metal powders in the composite sinter to give the conductive path for the feedthrough. However, unlike solid pins in traditional feedthrough they do not have high mass-density and electrical conductivity. High conductivity is desired in electrical feedthroughs in order to reduce the resistive losses of signal transfer, which lead to longer battery life. The other problem with the co-firing route is that the CMS based via forms spill over patterns between laminated green tapes or may even percolated into the ceramic insulation and can cause shorting of adjacent conductors. The ceramic during sintering shrinks around the solid metal conductor and the compressive force mechanically bonds the ceramics to the conductor. In such methods the solid metal conductors has the desired low electrical resistivity (˜1.1×10⁻⁸ Ωm). However, unlike the ceramic metal composite method and the traditional gold braze method, there is no chemical or metal bond between the conductor and the insulator, hermeticity of the joint maybe compromised. Hence there exists a need to increase the hermetic reliability for such feedthroughs. The current disclosure addresses this problem.

Co-pending application PCT/EP2019/060196 provides a feedthrough comprising a higher density of conductors. As a consequence of providing a higher density of conductors, the hermeticity of the feedthrough may be compromised, which for some critical applications such as those described herein may be insufficient. A higher density of conductors may result in micro-cracking adjacent to the conductors which results in reduced hermeticity and thereby a feedthrough that is deficient against performance criteria.

Hermeticity and performance of feedthroughs may be monitored using routine quality control testing leading to removal of the feedthrough if a reduction in the hermeticity or performance is detected. In order to avoid any unnecessary complications, such as repeat surgeries, it is desirable to produce a feedthrough device that provides improved hermeticity and overall performance more reliably.

The biocompatibility of implantable ceramic feedthroughs is provided by the chemical inertness of ceramic materials. However, the conductors in a feedthrough are often exposed outside of the chemically inert ceramic body, which is not electrically conductive, in order to enable further electrical connections to be made, for example, as wire bonding sites on the feedthrough. The inventors have found these regions of the feedthrough and interfaces between the conductors and the body to be particularly susceptible to leakages. Hence, feedthroughs are one of the most common failure points of high performance implantable devices that are required to be hermetic. It is a non-exclusive aim of the present disclosure to provide a hermetic feedthrough which is biocompatible and biostable, particularly for high density feedthroughs. It is also a non-exclusive aim of the present disclosure to meet the demanding package requirement for smaller-sized, high-density and hermetic feedthroughs.

### SUMMARY

In a first aspect of the present disclosure, there is provided a feedthrough assembly (1) comprising:
a feedthrough body (10) comprising: a ceramic body (2) having a first side (3) and a second side (4); a conductive element (5) extending through said ceramic body (2) between said first side (3) and said second side (4); and
a conductive pad (6) electrically connected to said conductive element (5);
   wherein the conductive pad (6) comprises a multi-layered arrangement comprising:
(i) a bonding layer (7) comprising one or more elements selected from the group consisting of Ti, Zr, Nb and V, said bonding layer in bonding contact with an end of the conductive element and the first or second side of the ceramic body; and
(ii) a diffusion barrier layer (8) disposed upon said bonding layer, comprising one or more elements selected from the group consisting of Nb, Ta, W, Mo and nitrides thereof, said diffusion layer having a different composition compared to the bonding layer; and/or
(iii) one or more sealing layers (9, 9a, 9b) disposed upon said bonding layer or said diffusion barrier layer.

In one embodiment, the one or more (or two more more) sealing layers each have a different composition compared to the bonding layer or the diffusion barrier layer. In another embodiment, adjacent layers within the multi-layered arrangement have a different composition to each other. In another embodiment, each layer of the conductive pad has a different composition. The composition of the each of the layers of the conductive pad may be different to the composition of the conductive element.

Use of “interconnect” is interchangeable with “conductive pad.” Reference to “feedthrough assembly” herein includes both an “feedthrough precursor” (e.g., 1.1 of FIG. 4), whose co-fired body and interconnect have not been sintered and a “finished feedthrough” (e.g., 1.2 of FIG. 5), whose co-fired body and interconnect have been sintered.

In a second aspect, there is provided a feedthrough precursor (1.1) comprising:
a feedthrough body (10) comprising: a ceramic body (2) having a first side (3) and a second side (4); a conductive element (5) extending through said ceramic body (2) between said first side (3) and said second side (4); and
a conductive pad (6) electrically connected to said conductive element (5);
wherein the conductive pad (6) comprises a multi-layered arrangement comprising:
(i) a bonding layer (7) comprising one or more elements selected from the group consisting of: Ti, Zr, Nb and V, said bonding layer in bonding contact with an end of the conductive element and the first or second side of the ceramic body; and
(ii) a diffusion barrier layer (8) disposed upon said bonding layer, comprising one or more elements selected from the group consisting of: Nb, Ta, W, Mo and nitrides thereof, said diffusion layer having a different composition compared to the bonding layer; and/or
(iii) one or more sealing layers (9a, 9b) disposed upon said bonding layer (7)8 or said diffusion barrier layer (8),
wherein upon sintering to form a feedthrough assembly, there is one or more of the following;
a helium leak rate of the feedthrough assembly decreases relative to the feedthrough precursor; preferably, the helium leak rate decreases by at least a factor of 10; an adhesive strength of the conductive pad on a side of the feedthrough assembly is increased;
the one or more sealing layers form an alloyed sealing layer; and/or
the one or more sealing layers partially diffuse into the diffusion barrier layer.

The feedthrough assembly of the present disclosure provides a conductive pad (also known as an interconnect pad) which is securely bonded to the at least one end of the conductive element with the bonding layer forming a bond with the end of the conductive element and a portion of an end of the ceramic body. The bonding layer preferably forms a reaction bond with the surface of the ceramic body, such that gaseous pathways emanating from the ceramic body, particularly proximal to the conductive element, are not able to escape through the conductive pad, which functions as a hermetic cap. The reliability and longevity of the bonding layer may be enhanced through the addition of a diffusion barrier layer, which functions to prevent the diffusion of components of the bonding layer away from a bonding surface comprising an end of the conductive element and an end (i.e. surface) of the ceramic body.

The bonding layer preferably comprises at least 50 wt % or at least 60 wt % of at least 70 wt % or at least 80 wt % or at least 90 wt % or 100 wt % of one of the following elements Ti, Zr, Nb and V. The bonding layer may further comprise minor components (e.g. less than 30 wt %) of Mo, Ta, W or Hf. In one embodiment the bonding layer comprises or consists of Ti.

In one embodiment, the diffusion barrier layer comprises at least 50 wt % or at least 60 wt % of at least 70 wt % or at least 80 wt % or at least 90 wt % or 100 wt % of one of the following elements Nb, Ta, W, Mo and nitrides thereof. In one embodiment the diffusion barrier layer comprises Nb, Ta, W, nitrides thereof or combination thereof. In another embodiment, the bonding layer comprises Nb, Ta and nitrides thereof or combination thereof. In a further embodiment, the diffusion barrier layer comprises Nb. In another embodiment, the diffusion barrier layer comprises Ta. In another embodiment, the diffusion barrier layer comprises W. In another embodiment, the diffusion barrier layer comprises Mo.

In embodiments, wherein the ceramic body comprises alumina, the bonding layer is preferably Ti. In some embodiments, wherein the ceramic body comprises zirconia, the bonding layer comprises W.

The diffusion layer has a different composition to the bonding layer. In particular, the diffusion barrier layer preferably has a different main elemental component to the bonding layer (i.e. the element making up the largest wt % of the diffusion barrier layer is different to the element making to the largest wt % of the bonding layer).

The diffusion barrier layer enables the sintering of the multi-layered assembly to occur without the unwanted diffusion of components between layers, which may compromise the functionality of the multi-layered assembly. The diffusion barrier layer may also function as a tie barrier to enable the one or more sealing layers to more securely adhere to the preceding layers of the conductive pad. Furthermore, by using thin film deposition techniques, the conductive pad does not significantly contribute to the resistivity of the feedthrough. In a preferred embodiment, the conductive elements are solid (e.g. wire or pin), thereby increasing the conductivity of the conductive pathway relative to assemblies comprising porous conductive elements, such as cermet.

The resistivity of the conductivity element and conductive pad is preferably no more than 1.0×10⁻⁴ Ω·cm or no more than 5.0×10⁻⁵ Ω·cm or no more than 1.0×10⁻⁵ Ω·cm. The increase in the resistivity of the conductivity pad, when connected to a conductive element is preferably no more than 50% or no more than 40% or no more than 30% or no more than 20% of the resistivity of the conductive element without the conductive pad.

Whilst there may be a degree of diffusion between the bonding layer and the diffusion barrier layer, the diffusion barrier layer preferably provides a continuous layer of one or more of Nb, Ta, W, Mo and nitrides thereof over the portion of the bonding layer that the diffusion barrier layer covers. The bonding layer and/or the diffusion barrier layer may comprise one or more sub-layers. The sub-layers may function to improve the adhesion between adjacent layers (e.g. improve the adhesion between the bonding layer and the diffusion barrier layer).

Other metals may be added to the bonding layer to form an alloy, however the proportion of Ti, Zr, Nb and V is preferably at least 10 wt % or at least 20 wt % or at least 30 wt % or in an amount sufficient to form a reaction bond with the surface of the ceramic body. The diffusion layer and sealing layer(s) may also comprise metal alloys. In another embodiment, the bonding layer and the diffusion barrier layer are deposited as substantially pure elemental layers.

In one embodiment, the bonding layer extends beyond the periphery of an end of the conductive element circumferentially, such that the minimum distance from the periphery of the bonding layer and the periphery of the conductive element is at least 1.0 μm or at least 2.0 μm or at least 5.0 μm or at least 10.0 μm or at least 20.0 μm or at least 40.0 μm or at least 80.0 μm. In some embodiments the minimum distance from the periphery of the bonding layer and the periphery of the conductive element is no more than 1 mm or no more than 400 μm or no more than 200 μm or no more than 100 μm or no more than 50 μm. The adjacent surface of the ceramic body is preferably substantially flush with the end of the conductive element. However, in some embodiments, the adjacent surface of the ceramic body may be configured to be at an offset level, above or below the height of the end of the conductive element.

The bonding layer preferably reacts with the ceramic body to form a strong reaction bond which may result in the transfer of oxygen from the ceramic substrate to the metal bonding layer resulting in oxygen deficient ceramic (e.g. alumina or zirconia) and an oxygen deficient metal oxide formed from the metallic bonding layer. This chemical reaction results in a stronger adhesive bond to the ceramic body than without the formation of the reaction bond. For example, a titanium bonding layer may react with a ceramic to form a reduced titania (TiO₂₋ₓ). Within limitations, the further the bonding layer extends beyond the periphery of the conductive element, the greater the bonding strength and hermeticity associated with the bonding layer as the overlap enables a hermetic bond to form between components and prevents the formation of gaseous pathways along the interface between the conductive pathway (5) and the ceramic body (2).

The extent that the bonding layer extends beyond the periphery of the conductive element may be limited by the proximity of neighbouring conductive elements. For high density feedthrough configurations, the extent at which the bonding layer extends beyond the periphery of the conductive element is preferably such that the distance between conductive pads is at least 10 μm or at least 20 μm or at least 30 μm or at least 50 μm. This distance provides a sufficient gap for each conductive pad to be electrically isolated from each other. In general, the greater the reaction bond area the greater gaseous resistance provided.

In another embodiment, the conductive pad extends no more than a distance equivalent to twice or thrice the diameter of the conductive element (5) and preferably no more than the diameter (or half the diameter) of the conductive element.

In one embodiment, the feedthrough comprises a plurality of conductive elements (5). The conductive element preferably have a density of conductive elements exceeding 1 conductor per 200,000 μm² or exceeding 1 conductor per 100,000 μm² or exceeding 1 conductor per 50,000 μm² or exceeding 1 conductor per 20,000 μm² or exceeding 1 conductor per 14,839 μm² (23 thou²) through a planar cross-section of the ceramic body. The present disclosure has been found to be particularly beneficial in maintaining hermeticity when applied to feedthroughs having a high density of conductive elements.

In other embodiments, the conductive pad further comprises one or more sealing layers, either disposed upon (i) said diffusion barrier layer or (ii) bonding layer. In some embodiments, the diffusion barrier layer may be omitted. The one or more sealing layers may provide a number of functional properties to the conductive pad, including passivation, anti-corrosive, wear resistance, tie layer (i.e. to enhance interlayer adhesion); gaseous barrier etc. However, a central focus of the one or more sealing layers is to enable the conductive pad to function as an interconnect and facilitate connection to other components within the electrical pathways of the device that the feedthrough assembly forms part of.

The one of more sealing layers may comprise one of more elements selected form the group consisting of Co, Ni, Al, Si, Cu, Ag, In, Cr, Ti, Ta, W, Mo, Au, Pt, Pd, Ni, Cr, Cu and Al. The one of more sealing layers may comprise Pt, Pd, Ni, Au, Cr, V, and combination thereof. In one embodiment, the one or more sealing layers comprise Pt, Pd, Ni, Au and combinations thereof. In some embodiments the one or more sealing layers comprises Ni comprises with at least one of Pt, Pt and Au. In one embodiment, the one of more sealing layers comprises a first sealing layer and a second sealing layer. The first sealing layer may function as a tie layer to enable the sealing layers to more securely bond to the diffusion barrier layer or the bonding layer.

The first sealing layer preferably comprises one or more elements selected from the group consisting of Co, Ni, Al, Si, Cu, Ag, In, Cr, Ti, Ta, W, Mo. The first sealing layer is preferably in contact with the diffusion barrier layer or the bonding layer.

The second sealing layer preferably comprises one or more elements selected from the group consisting of Au, Pt, Pd, Ni, Cr, Cu and Al, said second sealing layer having a different composition than said first sealing layer. The second sealing layer is preferably the top layer of the conductive pad. Upon sintering the first and second sealing layers may diffusion into each other and form a single alloy layer.

The number and combination of sealing layers will be dictated by the specific application. The one or more sealing layers may comprise a passivation layer to prevent electrical corrosion. In one embodiment, the first layer passivation layer comprises aluminium. Aluminium has a self-passivating surface and the ability to form an intermetallic phase with wire bonding metals (top/second layer) such as gold, copper and silver. In another embodiment, the first layer is a nickel layer which provides mechanical backing for a top gold layer, thereby improving wear resistance of the sealing layers.

It will be appreciated that the total number of layers in the multilayer arrangement may vary between at least 2 layers to typically no more than 10 layers and preferably no more than 6 layers or no more than 4 layers.

The skilled artisan will understand the various combinations of metallic layers which can be used to provide the required bonding to wire or other conductive pathways, having the required mechanical, corrosive resistance and conductive properties.

The ceramic body (2) may comprise advanced ceramic materials including but not limited to oxide or carbide or nitride ceramic materials. The ceramic body (2) may comprise ceramic-matrix composite materials. The ceramic body (2) may comprise alumina ceramics. The ceramic body (2) may comprise zirconia toughened alumina (ZTA) ceramics. The ceramic body (2) may comprise yttria-stabilized zirconia (YSZ) ceramics.

The thickness of the ceramic body will vary according to the application of the feedthrough, although the ceramic body thickness is typically between 0.5 mm and 50 mm or between 1.0 mm and 30 mm. The conductive pads of the present disclosure enable thinner ceramic body thicknesses to be achieved whilst maintaining excellent hermeticity. In some embodiments, the thickness of the ceramic body is less than 2.6 mm or less than 1.3 mm or less than 0.8 mm.

In one embodiment, the ceramic body preferably comprises at least 90.0 wt % or 95.0 wt % or at least 97.0 wt % or at least 98.0 wt % or at least 99.0 wt % or at least 99.5 wt % alumina or zirconia (and modified forms thereof). High purity alumina or zirconia is particularly preferred for interconnects used in medical applications.

The conductive element (5) may comprise Pt, Ir or combinations thereof. The conductive element (5) may comprise any other suitable conductive elements or materials. The conductive element (5) may be solid or porous and may comprise, including but not limited to, a solid rod, wire, lead, pathway, pin, metallic ink, cermet or via or another form of a conductor. The conductive pad may be advantageously applied to feedthroughs comprising a porous conducting element, such as cermet. Maintaining the required hermeticity on feedthrough comprising porous conductive element is typically difficult and the conductive pads of the present disclosure overcome some of the shortfalls of using porous conductive elements.

The conductive element (5) may comprise a plurality of conductive sub-elements (5a). The conductive pad (6) may be electrically connected to at least one of the conductive sub-elements (5a). The maximum linear length of the conductive pad (6) may be in the range of about 2 to about 100 times the diameter of each of the conductive sub-elements (5a) or conductive element (5).

The conductive element (5) may comprise at least a first end (14) proximal to said first side (3) of said ceramic body (2) and a second end (15) proximal to said second side (4) of said ceramic body (2). The first end (14) and the second end (15) of said conductive element (5) may be substantially parallel or flush with said first side (3) and said second side (4) of the ceramic body (2) respectively. The first end (14) and the second end (15) of said conductive element (5) may protrude out of said first side (3) and said second side (4) of the ceramic body (2) respectively. The first end (14) and the second end (15) of said conductive element (5) may be sunken into said first side (3) and said second side of the ceramic body (2) respectively.

The conductive pad (6) may provide a conductive pathway to said conductive element (5). The sub-elements (5a) may be in the form of a bundle of conductive elements which are housed within a single channel through the ceramic body (2) or plurality of conductive elements (5), with each conductive element housed within its own channel through the ceramic body (2). The conductive element is preferably has a diameter of between 10 μm and 100 μm.

The conductive pad (6) may act as an “interconnect” for further electrical connections to said conductive element (5). It will be understood that a second conductive pad (6b) may be provided on the second side (4) of the ceramic body (2) which is electrically connected to the conductive pad (6) via the conductive element (5).

The conductive element (5) may be brazed with the ceramic body (2) between the first side (3) and second side (4) forming a brazed interface (12a). The brazed interface (12a) may comprise a braze filler alloy comprising one or more elements selected from the list consisting of Au, Cu, Ag, Ti, Ni or combinations or alloys thereof. The brazed interface (12a) may further comprise of one or more elements originating from the ceramic body (2). The conductive element (5) may be in braze-less contact with the ceramic body (2) between the first side (3) and second side (4) forming a braze-less interface (12b). The braze-less interface (12b) may enable tighter spacing between said conductive element (5) and said ceramic body (2) due to the lack of a braze filler alloy.

The conductive pad (6) bonded to said first side (3) of said ceramic body (2) may provide a hermetic barrier over said first side (3) of said ceramic body (2). The conductive pad (6) bonded to said first side (3) of said ceramic body (2) may provide a hermetic barrier over the conductive element (5). The conductive pad (6) bonded to said first side (3) of the ceramic body (2) may provide a hermetic barrier over said first end (14). The conductive pad (6) bonded to said first side (3) of said ceramic body (2) may provide a hermetic barrier over said brazed interface (12a) or said braze-less interface (12b) between said conductive element (5) and said ceramic body (2).

The bonding layer (7) may have a density of at least 95% or at least 96% or at least 97% or at least 98% of the theoretical density of said bonding layer (7).

The feedthrough assembly (1) may comprise a He permeability of less than 1.0×10⁻⁷ cc·atm/s. The feedthrough (1) may have a He permeability of less than 1.0×10⁻⁸ cc·atm/s. The feedthrough (1) may have a He permeability of less than 1.0×10⁻⁹ cc·atm/s. The conductive pad (6) may provide the feedthrough (1) with a hermetic seal or a sintered seal over said first side (3) of the ceramic body (2). Reference to an increase in He hermeticity denotes a decrease in the permeability rate of He through the feedthrough assembly. The higher the hermeticity is, lower is the permeability.

In one embodiment a conductive pad (6) may be located between two adjacent components in the ceramic body. The component are preferably layers, although it will be appreciated that the ceramic body may be formed from other geometric configurations. The conductive pad may also extend between the adjacent components and function as a hermetic seal between conductive pathways in opposing components. Conductive pads may be located between several or all adjacent ceramic components in addition to, or as an alternative to, being located on an external surface of the ceramic body. Within this embodiment, holes are made in each of the green ceramic component and the holes filled with a conductive paste (e.g. metallic ink) to form a conductive pathway through the ceramic component.

The conductive paste preferably comprises a metallic conductor, such as a biocompatible metal (e.g. platinum group metal and alloys thereof). The paste may also comprise a binder (preferably a fugitive binder) and/or a ceramic filler to assist in matching the co-efficient of thermal expansion between the conductor and the ceramic body. A metal or metal alloy layer may then be coated over at least one end of the conductive pathway. The process may be repeated with one or more further metal/metal alloy components. The components may then be stacked or otherwise arranged such that the conductive pathway (5) extends from the first side (3) to the second side (4) of the ceramic body (2). In some embodiments, the conductive pathway may extend between the ceramic components as well as through the components. The assembly may be co-fired together such that the one or more conductive pads (6) form a bond between the ceramic components adjacent the conductive pathway. The formation of a feedthrough comprising a plurality of conductive pads between each side of the ceramic body is expected to provide even further enhancements in hermeticity. Further details of the formation of a feedthrough from a plurality of ceramic sheets is provided in U.S. Pat. No. 8,872,035.

The bonding layer metal element may react with the ceramic forming a chemical bond between the first side (3) of the ceramic body (2) and the bonding layer, at the joint interface (16). The bonding layer metal element may react with the first side (3) of the ceramic body (2) resulting in the formation of a reaction product. The reaction product may form as a continuous reaction layer at the joint interface (16)

The bonding layer (7) may comprise a reaction layer (17) proximal to the first side (3) of the ceramic body (2). The bonding layer metal element may be present in the reaction layer (17) in an amount ranging from about 70% wt to about 99.5% wt based on the total weight of the active metal component (i.e. metal component that reacts with the ceramic body to form the reaction layer) in the bonding layer. The reaction product may comprise but not be limited to an oxide, carbide, nitride, or silicide reaction product depending on the ceramic material selected and the reactions between the active alloy and the first side (3) of the ceramic body (2). The reaction layer (17) may comprise one or more elements originating from the bonding layer. The reaction layer (17) may comprise one or more elements originating from the ceramic body (2).

The reaction layer (17) may comprise one or more layers. The one or more layers may comprise a polycrystalline structure. The one or more layers may comprise one or more compounds.

The formation of the reaction layer (17) may depend on the chemical activity of the metal element in the metal or alloy used in the bonding layer. The chemical activity of the metal element may depend on the relative amounts of the metal element; the alloying elements (if used) and the chemical affinity between them. The chemical activity of the metal element may depend on the sintering temperature which provides a thermodynamic driving force for diffusion. The chemical activity of the metal element may depend on the sintering time which provides the time for diffusion to occur at the sintering temperature.

The reaction layer (17) may be continuous layer along the interface (16). The reaction layer (17) may add a higher degree of metallic character to the first side (3) of the ceramic body (2) enabling the active metal/alloy to wet and spread effectively over said first side (3) of the ceramic body (2). The chemical bond between the first side (3) of the ceramic body (2) and the bonding layer at the interface (16) may provide a hermetic seal or a sintered seal. The reaction layer (17) at the interface may provide a hermetic seal or a sintered seal.

The reaction layer (17) may be less than 10 μm thick or less than 5 μm thick or less than 3 μm thick. In one embodiment the thickness of the reaction layer (17) ranges from about 0.01 μm or 0.05 μm or 0.1 μm to 3 μm or 1 μm.

The conductive pad (6) may comprise one or more sealing layer(s) bonded to the diffusion barrier layer (8). The sealing layer(s) (9) may comprise one or more elements selected from the list consisting of Au, Pt, Ni, Cr, V, Cu, Ta, Ti, Nb, Al, Ag and Sn or combinations or alloys thereof.

The sealing layer(s) may function as a passivation barrier (e.g. when comprised of Au or Pt) and/or as a further bonding layer to connect the conductive pad to further conductive elements, such as wires or other components of an electrical circuit.

The first side of the ceramic body may be provided with a two or more precursor layers. The precursor layers are transformed into the bonding layer during the sintering step. For example, the bonding layer may comprise a layer of an active metal component and one or more layers of alloying components, which upon sintering form the bonding layer.

In one embodiment, the conductive pad (6, 6a) is derived from three or more layers (7, 8, 9a, 9b) comprising a first layer (7) bonded to the first side (3) of the ceramic body (2), a second layer (8) bonded on top of the first layer (7), a third layer (9a) bonded on top of the second layer (8); and a further layer (9b) bonded on top of the third layer.

The first layer (7) may comprise Ti. The second layer (8) may comprise Nb. The third layer (9a) may comprise Ni; and the fourth layer may comprise Au. Alternatively, the third layer and fourth layers may be interdisperse during the sintering process and form an Au—Ni alloy.

In one embodiment, the first layer (7) may have a thickness in the range of about 0.05 μm to 4 μm or 0.1 μm to about 2 μm, or about 0.2 μm to about 1.75 μm, or about 0.3 μm to about 1.5 μm. The second layer (8) may have a thickness in the range of about 0.1 μm to about 10 μm, or about 0.2 μm to about 5.0 μm, or about 0.3 μm to about 4.0 μm. The third layer (9a) may have a thickness in the range of about 0.1 μm to about 25 μm, or about 0.2 μm to about 15 μm, or about 0.3 μm to about 1.0 μm. The fourth layer (9b) may have a thickness in the range of about 0.1 μm to about 50 μm, or about 0.2 μm to about 20 μm, or about 0.3 micron to about 5.0 μm or about 0.4 μm to about 1.0 μm.

Depending upon the sealing/bonding technique employed to bond a wire to the conductive pad, the one or more sealing layers may an increased thickness to those embodiments stated above. An increased sealing layer(s) thickness may be preferred for some wire welding or soldering applications. In one embodiment, the one or more sealing layers has a greater thickness compared to the bonding layer and/or diffusion barrier layer thickness. In one embodiment, the ratio of the sealing layer(s) is between 1.5 to 100 times (or 3 to 50 times or 5 to 30 times or 10 to 20 times) greater than the thickness of the combined bonding and optional diffusion barrier layers.

The outer layer (9, 9b) may comprise a coating to provide a passivation layer over said bonding layer (7). The passivation layer may protect the conductive pad (6) thereby contributing to the hermeticity of the feedthrough (1).

The outer layer (9b) may fully encompass the preceding conductive pad layers (7, 8, 9a) so as to provide a protective shell to the conductive pad (6) that is hermetic to further enhance the hermetic seal. The outer layer (9b) may comprise Au and/or Pt to provide said passivation layer.

The outer layer (9b) may provide a conductive pathway to the conductive element (5) through the preceding layers in the conductive pad (6).

The outer layer (9b) may provide further electrical connections to be made, for example, the outer layer (9b) may provide a wire bonding site on the first side (3) of the ceramic body (2) for further electrical connections via said conductive pathway to the conductive element (5). As such the outer layer is conducive to be connected to further electrical connections through soldering, welding or other connection means.

The feedthrough (1) may comprise a second conductive pad (6a) electrically connected to said conductive element (5) wherein said second conductive pad (6) is bonded to said second side (4) of said ceramic body (2) through a bonding layer (7), said bonding layer (7) comprising a metal or alloy.

The second conductive pad (6a) may be electrically connected to the conductive pad (6) through said conductive element (5) thereby providing an electrical feedthrough with hermetic seals or sintered seals at both ends (14,15) of the conductive element (5).

The second conductive pad (6a) may comprise all embodiments of the conductive pad (6) as described herein. In one embodiment, the second conductive pad comprises a bonding layer comprising Ti; the diffusion barrier layer comprising Nb; and a sealing layer comprising Ni.

The feedthrough assembly (1) of the present disclosure may form part of an implantable medical device.

In a third aspect of the present disclosure, there is provided a method of producing a feedthrough assembly comprising a conductive pad comprising:
providing a feedthrough body (10) comprising:
   a ceramic body (2) having a first side (3) and a second side (4); and
   a conductive element (5) extending through said ceramic body (2) between said first side (3) and said second side (4);
optionally, machining an end of the conductive element and/or ceramic body, such that the end of the conductive element is substantially flush or otherwise offset with respect to with an adjacent surface of the ceramic body;
optionally, masking the area around the end of the conductive element, such that there is an unmasked area exposing the end of the conductive element and a portion of the adjacent surface of the ceramic body;
depositing a bonding layer to the end of the conductive element and the portion of the adjacent surface of the ceramic body, said bonding layer comprising one or more elements selected from the group consisting of Ti, Zr, Nb and V;
depositing
   a diffusion barrier layer on the bonding layer comprising one or more elements selected from the group consisting of Nb, Ta, W, Mo and nitrides thereof; and/or
   one or more sealing layers on the diffusion barrier layer or on the bonding layer; and
sintering at least the bonding layer to the ceramic body at sufficient temperature for the bonding layer to form a reaction bond with a surface of the ceramic body.

The presence of a reaction bond may be verified by an increase in adhesion between the bonding layer and the ceramic body after sintering.

The layers of the conductive pad may be sintered together or in multiple steps. For example, the bonding layer may first be sintered to the ceramic body, and then the diffusion barrier layer sintered to the bonding layer and then a sealing layer sintered to the diffusion barrier layer. Separate sintering, enables the sintering temperature to be optimised for each layer, thereby preventing excessive elemental diffusion during the sintering process.

Preferably, the ceramic body has been fired and more preferably the ceramic body and the conductive element have been co-fired together. With the provided feedthrough already co-fired (i.e. not green), the multi-layered conductive pad can be applied under less severe conditions enabling a conductive pad to be produced with:
lower porosity—resulting in increased conductivity;
smaller grain size—resulting in improved mechanical strength and hardness;
lower melting temperature metals—greater design flexibility;
lower roughness—increased dimensional tolerances;
thinner layers—increased conductivity and/or more compact design; and
greater positional accuracy—higher conductive element density.

The fired ceramic body may be polished to reduce the surface roughness enabling the multi-layered conductive pad to also have a reduced surface roughness compared to co-fired conductive pads. The Roughness (Rₐ) may be less than 2.0 μm or less than 1.5 μm or less than 1.0 μm or less than 0.5 μm or less than 0.3 μm. The Roughness (Rₘₐₓ) may be less than 5.0 μm or less than 3.0 μm or less than 2.0 μm or less than 1.0 μm or less than 0.5 μm.

The machining of the end of the conductive element and/or ceramic body preferably results in a roughness Rₐ of less than 100 μm or less than 50 μm or less than 5.0 μm or less than 3.0 μm or less than 2.0 μm or less than 1.0 μm or less than 0.5 μm.

The present disclosure increases the hermetic reliability of the feedthrough as well as acting as a pad for a stronger wire termination. The hermetic reliability is increased by creating an added barrier to the leak path (between the metal pin and the ceramic matrix). This layer is dense and bonds to the ceramic around the pin surface as well as the pin. Thus acting like a cap at both ends of the pin. Also because the pads are dense (because of the heat treatment post deposition) they present a sturdy surface for wire termination. Wire bonding technologies especially ultrasonic welding requires such sturdy interconnect pads for bond reliability and life.

The multi-layered arrangement preferably has a porosity of less than 5% v/v or less than 3% v/v or less than 2.0% v/v or less than 1.0% v/v or less than 0.5% v/v or less than 0.3% v/v. A lower porosity results in increased conductivity of the conductive pad compared to conductivity pads with higher porosity levels. For the purposes of the present disclosure, the ratio of void space (pores) to solid material may be taken to be the same as the surface area ratio of the void space to solid material as determined by image analysis software (e.g. ImageJ™).

Creating pads especially the gold and nickel layers through electroplating is also limiting. When the pads are too close to each other and the feature resolution is fine, electroplating leads to two issues. Firstly, the fine features are not well defined and may lead to shorting between pads, and two, there is an increase in defects and the plating peels off. Thus even when heat treated, some defects still remain added to some features that are not well defined. Therefore, the pads in the present disclose preferably created by a RF sputter method. This not only creates well defined features but also they are defect free and dense after heat treatment.

In one embodiment, the sealing layers comprise layers of gold and nickel which can be bonded to lead wires to connect both the circuitry in the can or leads to the nervous system, which can be made from platinum. Gold and nickel plating are very difficult to electroplate to feedthroughs with such close pin-to-pin spacing. The current disclosure overcomes this problem.

In embodiments wherein the conductive pad further comprising one or more sealing layers, these additionally layers are applied on top of the diffusion barrier layer. In some embodiments, the one or more sealing layers may be deposited after the sintering step, with an additional sintering step performed after the application of the one or more sealing layers. In other embodiments, a single sintering step is performed after the application of the bonding, diffusion barrier and one or more sealing layers. The sintering step assists in bonding the layers together and to the end of the conductive element and adjacent ceramic surface. In addition, the sintering step may densify the layers, thereby further enhancing the conductive cap's gas barrier properties.

In some embodiments, the machining of the conductive element and/or ceramic body results in the conductive element being counter-sunk into the ceramic body. Within this embodiment, the bonding layer may extend below a surface plane of the ceramic body and into a counter-sunk cavity. This configuration may result in a higher hermeticity due to the more tortuous gaseous pathway.

The unmasked area adjacent surface of the ceramic body is preferably an annular shape, with the layer extending by substantially even distance from the periphery of the conductive element.

The thickness of the bonding layer is in the range of 0.01 μm or 200 μm or 0.05 μm to 100 μm or 0.1 μm to 50 μm or 0.2 μm to 10 μm or 0.3 μm to 2.0 μm or 0.4 μm to 1.0 μm. The thickness of the diffusion barrier layer is in the range 0.05 μm to 200 μm or 0.10 μm to 100 μm or 0.1 μm to 50 μm or 0.2 μm to 20 μm or 0.3 μm to 10 μm or 0.4 μm to 2 μm or 0.5 μm to 1.0 μm. The thickness of the one of more sealing layers is in the range 0.1 μm to 500 μm or 0.05 μm to 200 μm or 0.1 μm to 100 μm or 0.2 μm to 50 μm or 0.3 μm to 20 μm or 0.4 μm to 10.0 μm or 0.5 μm to 2.0 μm or 0.6 μm to 1.0 μm. The thinner the layer the lower the resistivity the layers contributes to the conductive pad. However, the thickness of the layers have to be sufficient to enable a strong reaction bond with the ceramic surface and for the diffusion barrier layer to impede the mitigation of bonding layer components away from the ceramic surface.

In a one embodiment, the feedthrough may be formed from a larger co-fired monolithic block, multiple feedthroughs machined or sliced off the block to produce a plurality of feedthrough in which the conductive element is flush with the ceramic body at both ends of the feedthrough. Further details of this manufacturing technique is by provided in EP2437850, which is incorporated therein by reference.

The conductive pads of the present disclosure are particularly advantageous applied to co-fired feedthroughs which have been sliced into smaller feedthrough modules as the machining process can compromise the hermeticity of the co-fired feedthroughs through the generation of micro-cracks within the ceramic body. The use of the conductive pads of the present disclosure can not only restore the hermeticity of the feedthrough but further enhance the hermeticity as well as extending its durability.

In an alternative embodiment, a plurality of feedthrough sheets have conductive elements extending there through have conductive pads applied preferably one end, with the conductive pads extending beyond the peripheral of the conducive pads.

The thickness of the individual layers will depend upon the specific application, with thinner thicknesses favoured for feedthroughs used in implantable medical devices, whilst thicker layers may be favoured for industrial uses high mechanical resilience is required. The total thickness of the conductive pad may be in the range of 0.1 μm to 200 μm or 1 μm to 100 μm. In some embodiments, the conductive pad thickness may be less than 50 μm or less than 30 μm or less than 25 μm or less than 20 μm or less than 10 μm or less than 5.0 μm or less than 2.0 μm.

The thinner layers may be applied with any suitable technique, such as a thin-film deposition techniques, such as sputtering. These techniques are advantageously used with masking to enable the positioning of the layers to be tightly controlled, thereby enabling the conductive pads to the applied to high density feedthrough configurations. Greater layer thicknesses may be achieved using screen printing techniques or the like.

The application of the layers may be achieved using a thin film deposition technique such as sputter coating. The method of providing the bonding layer (7) to the first side (3) of the ceramic body (2) may comprise other thin film deposition techniques including but not limited to chemical vapour deposition, physical vapour deposition or screen printing or other thin film deposition techniques known in the art.

Sintering may be performed in a vacuum furnace at pressures ranging from about 4.0×10⁻⁴ to about 1.0×10⁻⁷ mbar. Sintering may be performed in a vacuum furnace at a pressure of less than about 1.0×10⁻⁵ mbar. Sintering may be performed in other chemically inert environments such as those comprising Ar or He or H gases or other chemically inert gases. The evacuation of oxygen in the chemically inert environment may promote diffusion of a metal element of the bonding layer (7) to the joint interface (16) to form a reaction bond (17).

The assembly may be heated at a heating rate ranging from about 1° C./min to about 15° C./min. The assembly may be heated to a sintering temperature for a predetermined time period or sintering time. The assembly may be first heated to a temperature below the sintering temperature for a predetermined time period in the range of between about 2 minutes to about 15 minutes to enable thermal homogenization of all components of the assembly.

The required sintering temperature will vary according to the composition of the bonding layer and the adjacent ceramic body. However, the temperature will be sufficient for the bonding layer to form a reaction bond to the adjacent surface of the ceramic body. This may be at a temperature below the melting point of the bonding layer. The sintering temperature is preferably at least 600° C. or at least 800° C. or at least 1000° C. or at least 1100° C. Preferably, the sintering temperature is sufficiently high enough to also sinter together the sealing and diffusion barrier layer to the bonding layer. Care should be taken to avoid excessive sintering temperatures which may result in excessive diffusion between the multi-layered conductive pad structure. The maximum sintering temperature is typically below the sintering temperature of the ceramic body and preferably no more than 1500° C. However, the specific sintering temperature and sintering time may be readily determined by a person skilled in the art of sintering. The sintering temperature may be selected to enable the diffusion of a metal element from the bonding layer into the joint interface (16) and reaction layer (17).

The sintering time may be in the range of about 1 minute to about 30 minutes, or about 2 minutes to about 25 minutes, or about 3 minutes to about 20 minutes. The sintering time may provide the time available at the sintering temperature for the metal element to diffuse to the joint interface (16). The sintering time may be selected to control the thickness of the reaction layer (17). The assembly may be cooled at a cooling rate ranging from about 1° C./min to about 10° C./min. A slow cooling rate is preferred to minimise thermally induced residual stresses that may be generated as a result of a coefficient of thermal expansion mismatch at the joint interface (16).

The method of producing a feedthrough assembly (1) may comprise a heat treatment comprising the steps of heating said feedthrough assembly (1). The heat treatment may be applied following sintering said bonding layer to said first side (3) of said ceramic body (2). The heat treatment may further densify the conductive pad (6). The heat treatment may further improve hermeticity of the feedthrough (1).

The average grain size after sinter and optional heat treatment is preferably less than 100 nm or less than 50 nm. The average gain size is smaller a co-fired conductive pad, thereby making the conductive pad relatively stronger than a co-fired version of the same conductive pad.

The method of producing a feedthrough assembly (1) may include pre-placing or depositing the bonding layer on the first side (3) of the ceramic body (2) to form an “assembly”. In some embodiments, the metal/alloy may be brushed or painted onto the first side (3) of the ceramic body (2), for example, in embodiments where the metal/alloy is in the form of a paste. The assembly may be subsequently mounted in a vacuum furnace for sintering. As will be appreciated by those skilled in the art, fixtures or fittings may be used to support the assembly during sintering and a load may be applied to secure said sintered assembly during sintering.

A method for connecting the conductive pad to a further electrical pathway may be achieved using a variety of possible bonding techniques including but not limited to welding, soldering, brazing, diffusion bonding, laser assisted diffusion bonding, laser welding, thermo-sonic bonding, ultrasonic bonding, soldering or flip chip bonding or other known joining techniques known in the art as will be appreciated by the skilled person.

For the purposes of the present disclosure, a layer represents a thin film of material which has a similar elemental composition (e.g. same main component). The elemental composition may vary within the layer, however there will be a discrete or transitional region which separates one layer from an adjacent layer. An elemental line scan (FIG. 8) may be one method to identify and differentiate the layers of the conductivity pad. Optical variations may also be used to distinguish between the layers of the conductive pad.

As noted previously, for the purposes of the present disclosure, reference to the feedthrough assembly includes reference to both a finished/sintered feedthrough and an unsintered feedthrough (“feedthrough precursor”), unless otherwise stated or otherwise apparent within the context of the specification.

Each of the bonding layer, diffusion barrier layer and one or more sealing layer may comprise a different composition and preferably each comprise a different main element (i.e. element having the highest concentration within the layer).

### BRIEF DESCRIPTION OF DRAWINGS

Embodiments will now be described, by way of example only and with reference to the accompanying drawings having like-reference numerals, in which:

FIG. 1 shows a schematic cross-sectional representation of the feedthrough assembly of the present disclosure in a first possible embodiment.

FIG. 2a shows a schematic cross-sectional representation of the feedthrough assembly of the present disclosure in a second possible embodiment.

FIG. 2b shows a schematic cross-sectional representation of the feedthrough assembly of the present disclosure in a third possible embodiment.

FIG. 3 shows a schematic cross-sectional representation of the feedthrough assembly of the present disclosure in a fourth possible embodiment.

FIG. 4 shows a schematic cross-sectional representation of the feedthrough assembly (1.1) of the present disclosure in a fifth possible embodiment.

FIG. 5 shows a sectional SEM micrograph of a portion of the feedthrough assembly (1.2) of the present disclosure corresponding to the fifth possible embodiment upon sintering.

FIG. 6 shows a magnified portion of the sectional SEM micrograph of FIG. 5.

FIG. 7a shows a photograph of a plurality of conductive pads comprising a Ti bonding layer and a Nb diffusion barrier layer according to a preferred embodiment of the present disclosure.

FIG. 7b shows a photograph of a plurality of conductive pads of FIG. 7a, with the further addition of Ni and Au sealing layers according to Example 1 of the present disclosure.

FIG. 7c shows a photograph of a cross-sectional view of the feedthrough assembly of Example 1.

FIG. 8 shows an EDS line-scan taken form the feedthrough assembly of FIG. 6.

### DETAILED DESCRIPTION OF A PREFERRED EMBODIMENT

The present disclosure provides an improved feedthrough device. The feedthrough may comprise assemblies comprising metal and ceramic components. The feedthrough may be used to transmit signals, high voltages, high currents, gases or fluids. The feedthrough may provide electrical insulation and high mechanical strength. The feedthrough may be hermetic and maintain ultra-high levels of vacuum and joint integrity that are maintained even at elevated temperatures, in cryogenic conditions, or in harsh environments such as in the human or animal body.

Sintering is one of the industrially preferred methods for coating ceramics whereby a metal/alloy is sintered at above 450° C. on a ceramic surface. The use of metal/alloys may result in the poor wetting of chemically inert ceramic surfaces and the generation of thermally induced residual stresses upon cooling due to a coefficient of thermal expansion mismatch at the ceramic-bonding layer interface which can cause the sintered coating to fail prematurely. As will be appreciated by the skilled person, the coating-ceramic interface comprises the interfacial region along the surfaces of two or more materials that are in contact or bonded together.

The present disclosure employs the use of a multi-layered conductive pad to overcome the abovementioned problems. Sintering using a multi-layered conductive pad structure enhances the capability of providing a durable and long lasting hermetic seal.

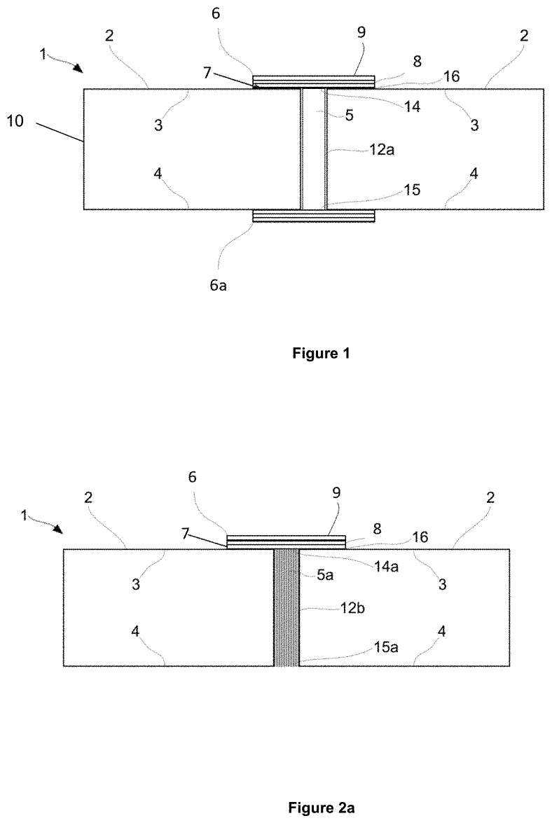

In accordance with embodiments of the disclosure, FIG. 1 shows a schematic cross-sectional representation of the feedthrough assembly (1) of the present disclosure in a first possible embodiment. The feedthrough assembly (1) comprises a feedthrough body (10) and a conductive pad (6). The feedthrough body (10) comprises: a ceramic body (2) having a first side (3) and a second side (4) and a conductive element (5) extending through said ceramic body (2) between said first side (3) and said second side (4). The conductive pad (6) is electrically connected to said conductive element (5) wherein the conductive pad (6) is bonded to said first side (3) of said ceramic body (2) through a bonding layer (7), with a diffusion barrier layer (8) provided to prevent the diffusion of components of the bonding layer from the joint interface (16) or the reactive layer (17), thereby weakening the adhesion of the conductive pad to the ceramic body. A further sealing layer (9) is provided to facilitate bonding to further electrical pathways that the feedthrough assembly may be connected to. An optional second conductive pad (6a) is similarly bonded on the second side (4).

In one embodiment, the ceramic body (2) comprises alumina, a cost-effective ceramic material with excellent refractoriness, electrical insulation, wear- and corrosion-resistance making it suitable for use in vacuum feedthroughs and high voltage insulation applications. In another embodiment, the ceramic body (2) comprises ZTA, providing excellent mechanical strength, wear-resistance, and toughness. In another embodiment, the ceramic body (2) comprises YSZ.

The ceramic material selected may depend on the application. For example, alumina may be selected for ultra-high vacuum coaxial feedthroughs used in signal transmission, particle physics, thin film deposition or ion beam applications due to excellent dielectric properties which provides high-voltage insulation with little signal attenuation. Optionally, the ceramic body (2) may comprise a polycrystalline or monocrystalline alumina.

The conductive pad (6) electrically connected to the conductive element (5) and bonded to the first side (3) of the ceramic body (2) has been found to improve hermeticity of the feedthrough (1). The conductive pad (6) is bonded to the first side (3) of the ceramic body (2) through a bonding layer (7). The bonding layer (7) comprises a metal or alloy that is capable for forming a reaction bond with the ceramic body. The overlaid diffusion barrier layer further enhances the hermetic seal through reducing gas permeability through the conductive pad (6) as well as improving the durability of the reaction bond through inhibiting diffusion of bonding layer components. The multi-layered arrangement of the provided by the conductive pad (6) provides a feedthrough assembly with improved hermeticity and performance while acting as an “interconnect” for further electrical connections to the conductive element (5).

The conductive element (5) may comprise any suitable conductive material such as Pt or Pt/Ir alloy. The conductive element (5) may comprise other conductive elements or materials. The conductive element (5) extends through the ceramic body (2) between said first side (3) and said second side (4).

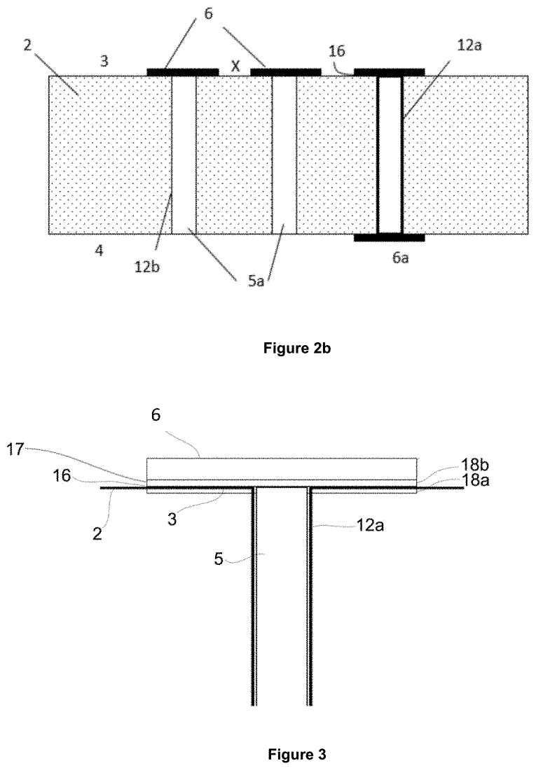

Referring to FIGS. 2a and 2b, in other embodiments, the conductive element (5) comprises a plurality of conductive sub-elements (5a). The plurality of conductive sub-elements (5a) may provide a densely packed feedthrough. The plurality of conductive sub-elements (5a) may provide a feedthrough (1) with one or more electrical conductors to increase the overall number of I/O signals as required for certain applications. The conductive pad (6) may be electrically connected to at least one of the conductive sub-elements (5a). Each of the plurality of conductive sub-elements (5a) may comprise one or more conductors with different properties, for example, a first pin comprising Pt, a second pin comprising Ir, and a wire comprising Pt and Ir.

Referring to FIGS. 1 to 2b, the conductive element (5) or plurality of conductive sub-elements (5a) extending through said ceramic body (2) between said first side (3) and said second side may comprise at least a first end (14, 14a) proximal to said first side (3) of said ceramic body (2) and a second end (15, 15a) proximal to said second side (4) of said ceramic body (2). In one embodiment, the first end (14, 14a) and the second end (15, 15a) of said conductive element (5) or plurality of conductive sub-elements (5a) is configured to be substantially parallel or flush with said first side (3) and said second side (4) of the ceramic body (2) respectively. The first end (14, 14a) and the second end (15, 15a) of said conductive element (5) or plurality of conductive sub-elements (5a) may be ground flat to be flush with said first side (3) and said second side (4) of the ceramic body (2) respectively. Optionally, the first end (14, 14a) and the second end (15, 15a) of said conductive element (5) or plurality of conductive sub-elements (5a) may protrude out of said first side (3) and said second side (4) of the ceramic body (2) respectively. Optionally, the first end (14, 14a) and the second end (15, 15a) of said conductive element (5) or plurality of conductive sub-elements (5a) may be sunken into said first side (3) and said second side of the ceramic body (2) respectively.

As illustrated in FIG. 2b, the feedthrough may comprise the plurality of conductive elements (5), with each conductive element (5) extending from a first side (3) to a second side (4) and being encompassed by said ceramic body (2).

In one embodiment, the conductive pad (6) provides a conductive pathway to the conductive element (5). In another embodiment, the conductive pad (6) provides a conductive pathway to a plurality of conductive sub-elements (5a). In a further embodiment, as will be discussed hereinafter, the feedthrough (1) may further comprise a second conductive pad (6a) electrically connected to said conductive element (5) wherein said second conductive pad (6b) is bonded to said second side (4) of said ceramic body (2). The conductive pad (6) may be electrically connected to the second conductive pad (6a) through said conductive element (5).

The conductive pad (6) acts as an “interconnect” for further electrical connections to said conductive element (5). In another embodiment, the conductive pad provides a first wire bonding site and a second conductive pad (6a) provides a second wire bonding site for further electrical connections to be connected to the feedthrough (1). The conductive pad (6) and the second conductive pad (6a) may each provide “interconnects” for further electrical connections to said conductive element (5).

In embodiments in which the further electrical connections are made to the conductive pad through mechanical connections, such as clamping, the bonding site preferably comprises a hard surface. Such hard surfaces may be obtained directly from the bonding layer or through the selection of an outer layer with the required hardness. In a particular, embodiment, the hard surface is formed from a multi-layered structure comprising a bonding layer and a diffusion barrier layer.

As will be appreciated by the skilled person, the conductive element (5) or the plurality of conductive sub-elements (5a) may be embedded in a ceramic matrix and compacted to form a green body that may subsequently be co-sintered to densify and impart mechanical strength to said green body compact forming a feedthrough (1) comprising the conductive element (5) or the plurality of conductive sub-elements (5a). The conductive pads (6) corresponding to respective conductive sub-elements (5a) are spaced apart by a gap (X) which corresponding to the location and size of the mask used when the conductive pad layers (6) were deposited.

In one embodiment, the conductive element (5) or the plurality of conductive sub-elements (5a) is brazed to the ceramic body (2) between the first side (3) and second side (4) forming a brazed interface (12a). The brazed interface (12a) may comprise a braze filler alloy comprising one or more elements selected from the list consisting of Au, Cu, Ag, Ti, Ni or combinations or alloys thereof. The brazed interface (12a) may further comprise of one or more elements originating from the ceramic body (2). In another embodiment, the conductive element (5) or the plurality of conductive sub-elements (5a) is in braze-less contact with the ceramic body (2) between the first side (3) and second side (4) forming a braze-less interface (12b). The braze-less interface (12b) may enable tighter spacing between said conductive element (5) and said ceramic body (2) due to the lack of a braze filler alloy. Optionally, the braze-less interface (12b) may enable tighter pin-to-pin spacing between said plurality of conductive sub-elements (5a) due to the lack of a braze filler alloy.

The conductive pads (6, 6a) provide a hermetic barrier or hermetic seal, an airtight seal that may prevent the passage of air, oxygen, or other gases. The hermeticity, or leak-tightness, of a component may be tested using a variety of methods known in the art including leak testing. Leak testing is a non-destructive method used to locate and measure the size of leaks into or out of a component under vacuum or pressure. A tracer gas is introduced to the component connected to a leak detector. Helium leak testing is an effective test method for hermeticity due to the relatively small atomic size of helium atoms which may easily pass through any leaks in the component. Leak rates with a He hermeticity as low as 1.0×10⁻¹⁰ cc·atm/s may be detected. For example, for a component required to be watertight, a leak rate with a He hermeticity of 1.0×10⁻⁴ cc·atm/s would be sufficient. During a helium leak test, a pressure difference between an inner side and an outer side of a component under examination is produced.

In some embodiments, the conductive pad (6) has a Mohs hardness of at least 2.5 or at least 3.0 or at least 3.5 or at least 4.0 or at least 4.5. A high hardness value enables mechanical connections to be made, such as further electrical connections mechanically clamped to the first wire bonding site provided by the top surface of the conductive pad (6). In applications requiring mechanical connections, the properties of the diffusion barrier layer (8) including hardness and strength may be sufficient without the need of a separate outer sealing layer(s) (9), as will be described hereinafter.

The bonding layer may comprise alloying elements may host an active metal element in an active alloy. The alloying elements may facilitate or promote the diffusion of an active metal element to the first side (3) of the ceramic body (2) in the formation of a hermetic seal. The alloying elements may facilitate or promote the diffusion of the active metal element to the joint interface (16) in the formation of a hermetic seal.

The alloying elements may comprise one or more elements with a low “chemical affinity” towards the active metal element. As will be appreciated by the skilled person, the low chemical affinity may comprise a low solubility to form phases or a low tendency to form compounds between the active metal element and the alloying elements.

The active metal element in the bonding layer may be selected depending on the ceramic material to be sintered, for example, Ti may be selected for an alumina ceramic body (2). The active metal element selected may depend on the metal or alloying elements in the bonding layer and the chemical affinity between said active metal element(s) so as not to inhibit the diffusion of said active metal element to the joint interface (16) in the formation of a hermetic seal or active sintered seal.

The active metal element or the alloying elements selected in forming a suitable active bonding layer may further depend on the physical properties of the active alloy desired, such as strength, hardness, coefficient of thermal expansion, liquidus temperature, corrosion resistance, biocompatibility and electrical conductivity.

The bonding layer may comprise one or more active metal elements or one or more alloying elements to provide an alloy having a eutectic temperature so as to enable a reduced sintering temperature. The alloying elements may form an alloy having a eutectic temperature thereby enabling a reduced sintering temperature. A reduced sintering temperature may help to minimise the generation of thermally induced residual stresses due to a coefficient of thermal expansion mismatch at the joint interface (16).

In some embodiments, the bonding layer may be derived from a layered structure having one or more layers. Each layer may comprise different metals that have a eutectic temperature when formed into an alloy during the sintering process.

Referring to FIG. 3, in another embodiment, the bonding layer (7) comprising an active braze alloy comprises a reaction layer (17) proximal to the first side (3) of the ceramic body (2) having one or more layers (18).

In one embodiment, the one or more layers (18) comprises a first layer (18a) and a second layer (18b), the first layer (18a) is proximal to the first side (3) of the ceramic (2) body and the second layer (18b) is bonded on top of the first layer (18a). In another embodiment, the reaction layer (17) comprises the first layer (18a). In another embodiment, the reaction layer (17) comprises the second layer (18b). For example, in some embodiments, the ceramic body (2) comprises an alumina ceramic and the bonding layer comprises an active metal element and alloying elements. The alloying elements comprises an Ag—Cu eutectic alloy with around 72% wt Ag and around 28% wt Cu. In one embodiment, the active metal element comprises Ti in the range of about 1.75 to about 4.5% wt. The reaction layer (17) comprises the first layer (18a) comprising a thin (e.g. nanometer(s) thick) TiO layer and the second layer (18b) comprising a Ti₃Cu₃O. In another embodiment, the active metal element comprises Ti in the range of less than 1.75% wt. The reaction layer (17) comprises the first layer (18a) comprising a thin TiO layer. In another embodiment, the active metal element comprises Ti in the range of at least 4.5% wt. The reaction layer (17) comprises the second layer (18b) comprising Ti₃Cu₃O.

### Example 1

A co-fired alumina Pt/Ir (diameter 50.8 μm) feedthrough was diced (1 mm thickness) from a larger block and subsequently ground and lapped flat with Rₐ being less than 10 μm finish.
1. Mask the feedthrough such that only the area of the proposed conductive padding is exposed over the pin for sputtering.
2. Deposit a titanium layer of approximately 400 nm thickness on top a pin and extending radially approximately at least 100 μm onto the top of the ceramic substrate.
3. Deposit a niobium layer of approximately 2.0 μm thickness by sputtering.
4. Deposit a nickel/chrome (80/20) layer of approximately 1 μm thickness by sputtering.
5. Sputter coat a final layer of gold of approximately 0.5 μm thickness.
6. Sinter the assembly at 1100° C. for approximately 30 minutes.

A variation of the above methodology is to first sinter the niobium and titanium layers at 1100° C. for approximately 30 minutes, prior to sputter coating the third and fourth layers after which the assembly is sintered at 950° C. for approximately 10 minutes.

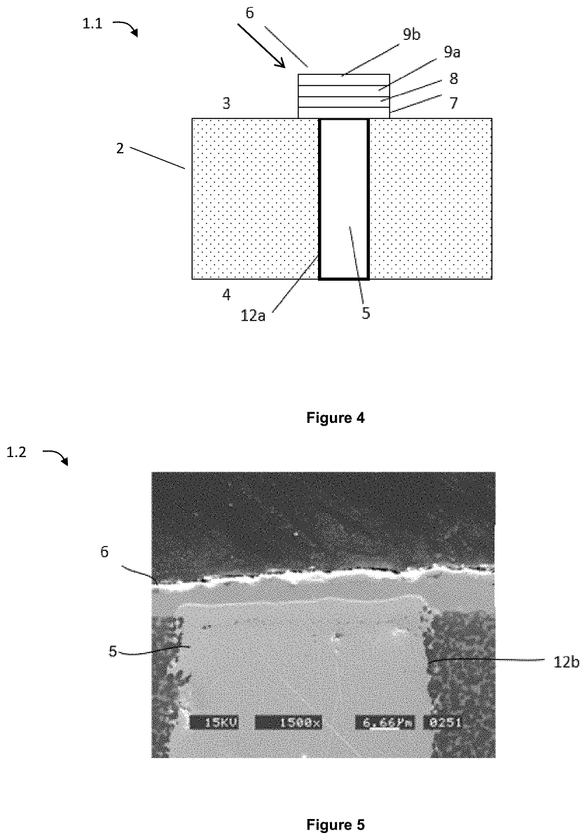

A schematic diagram of the layer structure of the feedthrough precursor (1.1) to the feedthrough in the above mentioned example is provided in FIG. 4, the first side (3) of the ceramic body (2) is provided with a multi-layered conductive pad (6) prior to sintering. The bonding layer (7) comprises Ti; the barrier diffusion layer (8) comprises Nb; the first sealing layer (9a) comprises Ni and the second (top) sealing layer (9b) comprises Au. After sintering, the first and top sealing layers (9a and 9b, respectively) may disperse into one another to form a single Au—Ni alloy layer. After sintering, there is partial diffusion of the bonding layer (7) into the barrier diffusion layer (8).

FIG. 5 is a sectional scanning electron microscope (SEM) micrograph showing a cross section of the finished feedthrough (1.2) according to the configuration illustrated in FIG. 4 after the sintering step. The finished feedthrough (1.2) comprises the conductive element (5), the conductive pad (6), and the braze-less interface (12b). The Roughness (Rₘₐₓ) of the conductive pad is estimated to be less than 1.0 μm.

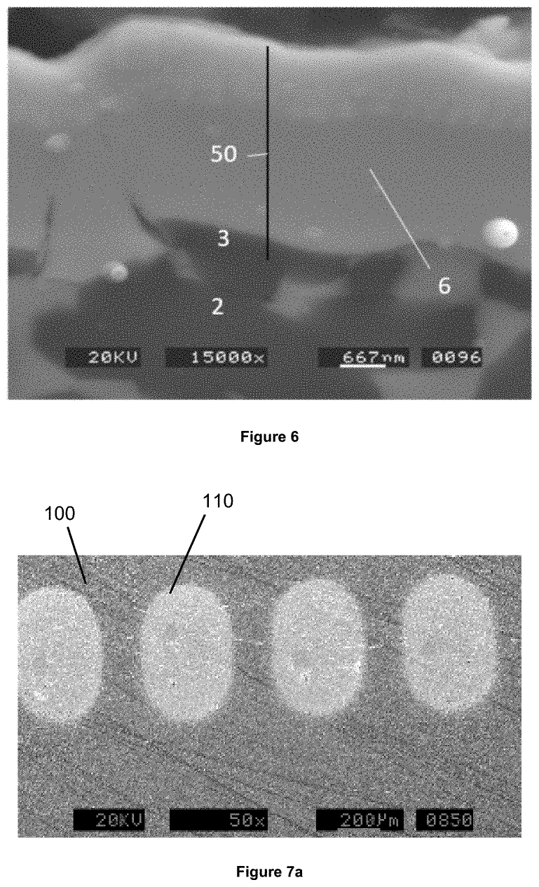

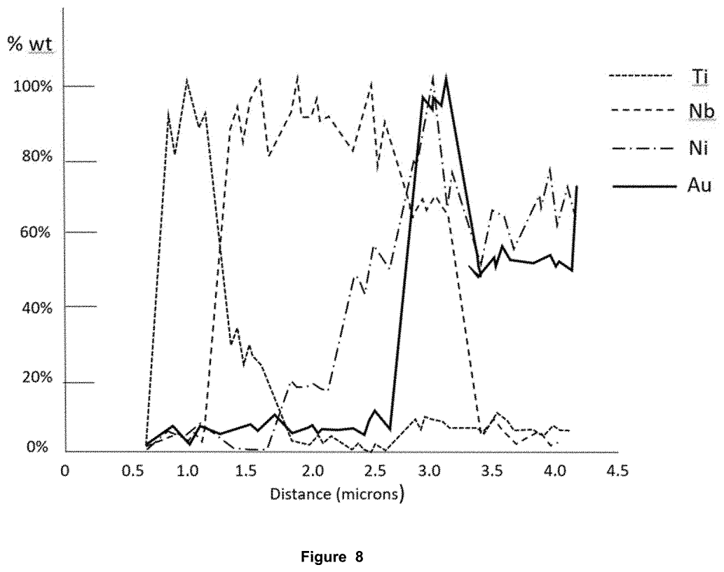

FIG. 6 illustrates a portion of the conductive pad (6) reaction bonded to the surface of the first side (3) of the ceramic body (2). An EDS line-scan (50; FIG. 8) revealed that the Ti bonding layer (7) was approximately 400 nm thick and the Nb diffusion barrier layer (8) was about 2 μm thick. The line-scan also reveals that there was a small amount of diffusion of titanium into the diffusion barrier layer (e.g. <less than about 500 nm) before the titanium intensity levels reached a background noise level, signifying no detectable titanium levels. Without the diffusion barrier layer, the titanium bonding layer and sealing layers are likely to have diffused into each other, weakening the bond or the longevity thereof, between the bonding layer and the ceramic substrate.

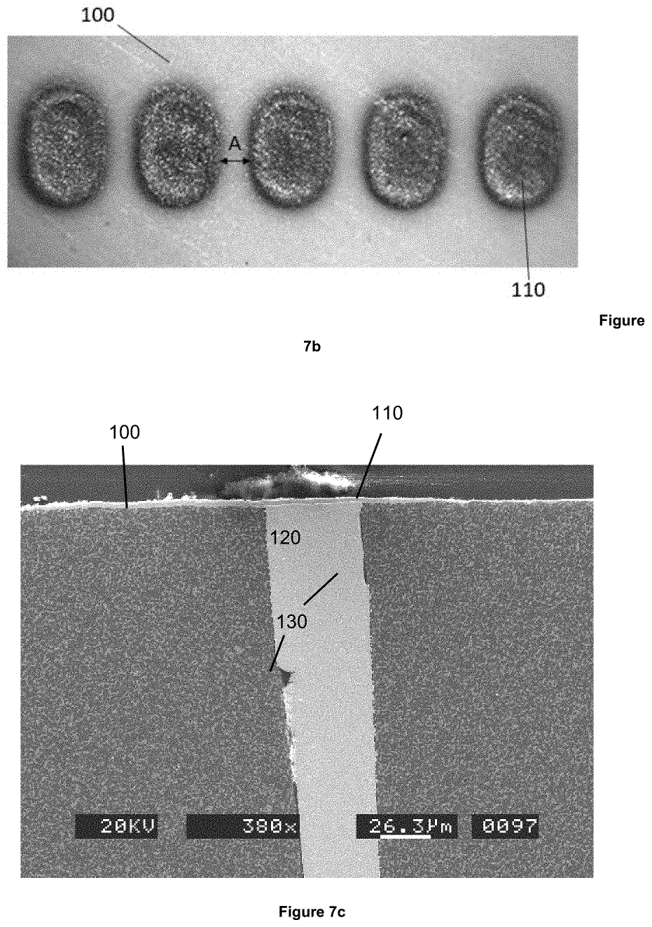

FIG. 7a illustrates the top view of the feedthrough assembly (110) after the Ti and Nb has been deposited over the conductive element and adjacent ceramic body (100). FIG. 7b illustrates the top view of the completed sintered feedthrough assembly 110. FIG. 7c is a cross-section view of one of the conductive elements (120) with the conductive pad (110) covering both the top of the conductive element and the surface of the ceramic body (100). The interface 130 between the conductive element and the ceramic body comprises void spaces, formed during the co-firing process, which may enable gas to leak through the feedthrough. The conductive pad, with its secure bond to the surface of the ceramic body provides additional protection against gaseous leaks.

The line-scan (FIG. 8) also reveals that the first sealing layer (9a) and the second sealing layer (9b) have diffused into each other to form a single Ni—Au alloy sealing layer having a thickness of about 1.5 μm. The line-scan also reveals a degree of diffusion of nickel and gold into the niobium diffusion barrier layer.

### Hermeticity

The hermeticity tests were performed on nine samples of the feedthrough with and without a conductive pad. The conductive pad was derived from a four layer assembly structure as represented in FIG. 4 which was sintered to produce the feedthrough assembly of FIG. 5. The feedthroughs were tested for hermeticity using the protocol of MIL-STD-883 test method 1014 and test condition. Table 1 shows the results of hermeticity testing performed on nine samples of this embodiment, according to the method discussed herein.

TABLE 1

Helium leak rate (cc · atm/s)

| Sample | Without conductive pad | With conductive pad |
|---|---|---|
| 1 | 6.4 × 10⁻¹⁰ | 8.2 × 10⁻¹¹ |
| 2 | 5.2 × 10⁻⁹ | 3.1 × 10⁻¹⁰ |
| 3 | 1.3 × 10⁻⁹ | 6.1 × 10⁻¹¹ |
| 4 | 1.9 × 10⁻¹⁰ | 2.2 × 10⁻¹⁰ |
| 5 | 4.2 × 10⁻⁶ | 3.1 × 10⁻⁸ |
| 6 | 3.9 × 10⁻⁷ | 1.6 × 10⁻⁸ |
| 7 | 8.2 × 10⁻⁶ | 3.3 × 10⁻⁹ |
| 8 | 7.1 × 10⁻⁶ | 2.4 × 10⁻⁹ |
| 9 | 4.8 × 10⁻⁶ | 3.1 × 10⁻⁸ |
| Average | 2.7 × 10⁻⁶ | 9.4 × 10⁻⁹ |

The hermeticity tests were subsequently repeated after the conductive pad was bonded to the first side of said ceramic body. The results showed that the conductive pad provided the feedthrough with an improved hermetic seal or a sintered seal over said first side of the ceramic body. For each sample, an increase in the He hermeticity (reduction in He permeability) was observed. The average He hermeticity increased from 2.7×10⁻⁶ cc·atm/s to 9.4×10⁻⁶ cc·atm/s for the nine samples.

### Resistivity

The resistivity (at room temperature) of the feedthrough of Example 1 was measured with and without the conductive pad, with the results (Table 2), confirming that the conductive pad is able to maintain a high conductivity of the feedthrough assembly.

TABLE 2

| | | | % |
|---|---|---|---|
| | Pt/Ir (90/10) | +conductive pad | change |
| Average Resistivity (Ω · cm) | 2.78 × 10⁻⁵ | 3.79 × 10⁻⁵ | 36 |
| Standard Deviation (Ω · cm) | 3.65 × 10⁻⁶ | 9.10 × 10⁻⁶ | — |

### Effect of the Sintering Step

As illustrated in FIGS. 7a b & c, a feedthrough assembly was formed according to the procedure of Example 1, with a co-fired zirconia toughened alumina substrate (100) with five 50 μm diameter Pt/Ir pins with a centre to centre spacing of approximately 620 μm. The ceramic substrate was approximately 1 mm thick and machined from a monolithic feedthrough block. Each of the pins had an oblong conductive pad sputtered coated and sintered. The estimated roughness (Rₘₐₓ) of the conductive pad is estimated to be less than 1.0 μm.

Each oblong shaped conductive pad had a width of approximate 420 μm (radial overlap of approximately 185 μm) and a length of approximately 800 μm (i.e. 375 μm radial overlap). The gap “A” between adjacent conductive pads was approximately 200 μm.

The second side was sputter coated and sintered with the oblong shaped conductive pad comprising the same thickness and diameter layers of Ti and Nb, followed by a Ni/V alloy coating layer (75 nm) and a 450 nm Au top coating.

A hermeticity test was performed on the feedthrough before and after the sintering step, with the results provided in Table 3. The results indicate that sintering significantly reduces the amount of helium which leaks through the feedthrough. The decrease in the helium leakage may be attributable to the reaction bond layer created at the ceramic-Ti interface, in addition to the sintering step densifying the layers of the conductive pad.

TABLE 3

Helium leak rate (cc · atm/s)

| Sample | No sintering | First side sintered |
|---|---|---|
| 1 | 1.6 × 10⁻⁸ | 1.7 × 10⁻¹¹ |
| 2 | 6.4 × 10⁻⁹ | 8.8 × 10⁻¹¹ |
| 3 | 2.4 × 10⁻¹⁰ | 3.6 × 10⁻¹⁰ |
| Average | 1.0 × 10⁻⁹ | 3.6 × 10⁻¹¹ |

The conductive pads were also evaluated for adhesion to the ceramic surface. When adhesive tape was applied and removed from the unsintered first side of the feedthrough a substantial proportion of the conductive pads were observed to be removed with the adhesive tape. However, there was no removal of the conductive pads when the adhesive tape was applied to the sintered first side of the feedthrough. The sintered conductive pad were then resistance welded to gold wires. Tweezers were used to assess the strength of the bond, with the bond strength deemed excellent. The test results confirm the presence of a reaction bond between the bonding layer and the ceramic substrate.

1. A feedthrough assembly comprising:
a feedthrough body comprising: a ceramic body having a first side and a second side; a conductive element extending through said ceramic body between said first side and said second side; and
a conductive pad electrically connected to said conductive element;
wherein the conductive pad comprises a multi-layered arrangement comprising:
(i) a bonding layer comprising one or more elements selected from the group consisting of Ti, Zr, Nb and V, said bonding layer in bonding contact with an end of the conductive element and the first side or second side of the ceramic body; and
(ii) a diffusion barrier layer comprising one or more elements selected from the group consisting of Nb, Ta, W, Mo and nitrides thereof, said diffusion layer having a different composition compared to the bonding layer; and/or
(iii) one or more sealing layers disposed upon said bonding layer or said diffusion barrier layer.

2. The assembly according to claim 1, wherein the multi-layered arrangement comprises the one or more sealing layers, disposed upon said diffusion barrier layer, said one or more sealing layers each having a different composition compared to the diffusion barrier layer.

3. The assembly according to claim 1, wherein the multi-layered arrangement comprises one or more sealing layers, disposed upon said bonding layer, said one or more sealing layers each having a different composition compared to the bonding layer.

4. The assembly according to claim 1, wherein the one of more sealing layers comprises one or more elements selected from the group consisting of Pt, Au, Ni, Pd, Cr, V, and Co.

5. The assembly according to claim 1, wherein the bonding layer further comprises one or more elements selected from the group consisting of Mo, Ta, W and Hf.

6. The assembly according to claim 1, wherein the bonding layer comprises Ti.

7. The assembly according to claim 1, wherein the diffusion barrier layer comprises one or more elements selected from the group consisting of Nb, Ta, W and nitrides thereof.

8. The assembly according to claim 7, wherein the diffusion barrier layer comprises Nb or nitrides thereof.

9. The assembly according to claim 1, wherein the bonding layer comprises Ti; the diffusion barrier layer comprises Nb; and the one or more sealing layers comprises Ni and Au.

10. The assembly according to claim 9, wherein the assembly comprises a second conductive pad electrically connected to the conductive element on the opposing side of the ceramic body, said second conductive pad comprising a bonding layer comprising Ti; a diffusion barrier layer comprising Nb; and a sealing layer comprising Ni.

11. The assembly according to claim 1, wherein the resistivity of the conductivity element and conductive pad is no more than 5.0×10⁻⁵ Ω·cm.

12. The assembly according to claim 1, wherein the bonding layer and/or the diffusion barrier layer has a thickness in the range of 0.01 μm to 10 μm.

13. The assembly according to claim 1, wherein the one or more sealing layers have a thickness between 1.5 to 100 times greater thickness than the combined thickness of the bonding layer and the diffusion barrier layer.

14. The assembly according to claim 1, wherein the density of the conductive elements exceeds 1 conductor per 100,000 μm² through a planar cross-section of the ceramic body.

15. The assembly according to claim 1, wherein said feedthrough upon sintering has a He permeability of less than 1.0×10⁻⁷ cc·atm/s.

16. A medical device feedthrough comprising the assembly according to claim 1.

17. A method of producing a feedthrough assembly comprising:
providing a feedthrough body comprising:
a ceramic body having a first side and a second side; a conductive element extending through said ceramic body between said first side and said second side;
optionally, machining an end of the conductive element, such that the end of the conductive element is substantially flush or otherwise offset with respect to an adjacent surface of the ceramic body;
optionally, masking the area around the end of the conductive element, such that there is an unmasked area exposing the end of the conductive element and a portion of the adjacent surface;
depositing a bonding layer to the an end of the conductive element and a portion of the adjacent surface of the ceramic body, said bonding layer comprising one or more elements selected from the group consisting of Ti, Zr, Nb, Ta, V, Mo, Mn, W, Y and Hf and combinations thereof;
depositing a diffusion barrier layer on the bonding layer comprising one or more elements selected from the group consisting of Nb, Ta, W, Mo and nitrides thereof; and/or one or more sealing layers on the diffusion barrier layer or on the bonding layer; and
sintering at least the bonding layer to the ceramic body at a sufficient temperature for the bonding layer to form a reaction bond with a surface of the ceramic body.

18. The method according to claim 17, wherein the feedthrough body has been fired prior the depositing of the bonding layer.

19. The method according to claim 17, wherein the one or more sealing layers are deposited after Step F, and the one or more sealing layers are sintered at sufficient temperature and time for the one or more sealing layers to bond to the adjacent layer(s).

20. A feedthrough assembly produced by the method of claim 17.

21. A feedthrough precursor comprising:
a feedthrough body comprising: a ceramic body having a first side and a second side; a conductive element extending through said ceramic body between said first side and said second side; and
a conductive pad electrically connected to said conductive element;
wherein the conductive pad comprises a multi-layered arrangement comprising:
(i) a bonding layer comprising one or more elements selected from the group consisting of Ti, Zr, Nb and V, said bonding layer in bonding contact with an end of the conductive element and the first side or second side of the ceramic body; and
(ii) a diffusion barrier layer comprising one or more elements selected from the group consisting of Nb, Ta, W, Mo and nitrides thereof, said diffusion layer having a different composition compared to the bonding layer; and/or
(iii) one or more sealing layers disposed upon said bonding layer or said diffusion barrier layer,
wherein upon sintering to form a feedthrough assembly, a helium leak rate of the feedthrough assembly decreases relative to the feedthrough precursor.

22. The feedthrough precursor of claim 21, wherein upon sintering to form the feedthrough assembly, the helium leak rate decreases by at least a factor of 10.
